(19) United States
(12) Patent Application Publication
(10) Pub. No.: US 2026/0157751 A1
(43) Pub. Date: Jun. 11, 2026

(54) SURGICAL INSTRUMENT

(71) Applicant: Arthrex, Inc., Naples, FL (US)

(72) Inventors: Joseph P. Aguila, Naples, FL (US); Jason Valentin, Fort Myers, FL (US)

(73) Assignee: Arthrex, Inc., Naples, FL (US)

(21) Appl. No.: 19/414,534

(22) Filed: Dec. 10, 2025

Related U.S. Application Data
(60) Provisional application No. 63/730,144, filed on Dec. 10, 2024.

(51) Int. Cl. A61B 17/04

(52) U.S. Cl. CPC A61B 17/0469

(57) ABSTRACT

The surgical instrument includes a proximal end and a distal end. The distal end is opposite the proximate end and is defined by a tube. The tube defines at least a first member and a second member. The first member and the second member are movable between a first state and a second state, and in the second state, the first member is spaced apart from the second member.

### CROSS-REFERENCE TO RELATED APPLICATION

This application priority to and the benefit of U.S. Provisional Application 63/730,144 filed on December 10, 2024. The disclosure of the above application is incorporated herein by reference.

### BACKGROUND

The present disclosure relates to medical devices, and more particularly relates to a surgical instrument, such as a grasper.

Surgery may be required to reattach the soft tissue to the bone to promote healing of the soft tissue and corresponding joint(s). In certain instances, the

surgery may be performed arthroscopically, using small incisions to access a region within an anatomy of a patient proximate the corresponding joint(s).

### SUMMARY

This section provides a general summary of the disclosure and is not a comprehensive disclosure of its full scope or all of its features.

The present disclosure provides a surgical instrument for the repair of soft tissue. The surgical instrument may generally include a distal end that may be movable between a first state and a second state.

In one example, the surgical instrument includes a proximal end and a distal end. The distal end is opposite the proximate end and is defined by a tube. The tube defines at least a first member and a second member. The first member and the second member are movable between a first state and a second state, and in the second state, the first member is spaced apart from the second member.

In one example, the first member cooperates with the second member to define a slot in the first state. The first member has a lip at an end that extends beyond a second end of the second member. The first member includes a projection proximate the lip, which is received within a groove defined in the second member in the first state to define a terminal end of the slot. The first member includes a first mating surface, the second member includes a second mating surface, and the projection is defined in the first mating surface and the groove is defined in the second mating surface. The first mating surface faces the second mating surface and cooperates with the second mating surface to define the slot in the first state. The distal end includes a plurality of struts, and at least a pair of the plurality of struts cooperate to enclose an end of the slot.

The distal end includes a plurality of ribs, with each rib associated with at least one strut of the plurality of struts. Each of the plurality of struts has an omega shape, with an apex and a pair of legs that define a base. The second end of the distal end is open about a perimeter of the distal end.

A method is provided for soft tissue repair, which includes inserting the surgical instrument into an incision in an anatomy. The method includes directing the surgical instrument to a predetermined location within the anatomy. The surgical instrument includes a distal end defining a first member and a second member. The method includes moving the first member and the second member from the first state to the second state such that the first member is spaced apart from the second member. The method includes moving the surgical instrument to at least partially surround a portion of a medical device. The method includes moving the first member and the second member from the second state to the first state to retain the portion of the medical device between the first member and the second member. The method includes removing the surgical instrument from the anatomy while retaining the portion of the medical device to repair soft tissue.

A kit including the surgical instrument is also provided.

Also provided is a surgical instrument. The surgical instrument includes a proximal end and a distal end opposite the proximal end. The distal end includes a tube that defines at least a first member and a second member. The first member and the second member are movable between at least a first state and a second state. The first member cooperates with the second member to define a slot in the first state and in the second state the first member is spaced apart from the second member.

The first member has a lip at an end that extends beyond a second end of the second member. The first member includes a projection proximate the lip, which is received within a groove defined in the second member in the first state to define a terminal end of the slot. The first member includes a first mating surface, the second member includes a second mating surface, and the projection is defined in the first mating surface and the groove is defined in the second mating surface. The first mating surface faces the second mating surface and cooperates with the second mating surface to define the slot in the first state. The second end of the distal end is open about a perimeter of the distal end. The distal end includes a plurality of struts, and at least a pair of the plurality of struts cooperate to enclose an end of the slot. The distal end includes a plurality of ribs, with each rib associated with at least one strut of the plurality of struts.

Further provided is a surgical instrument that includes a proximal end and a distal end opposite the proximal end. The distal end includes a tube that defines at least a first member and a second member. The first member includes a lip at an end that extends beyond a second end of the second member and a projection proximate the lip. The first member and the second member are movable between at least a first state and a second state. The first member cooperates with the second member to define a slot in the first state with the projection received within a groove defined in the second member in the first state to define a terminal end of the slot. In the second state, the first member is spaced apart from the second member.

The first member includes a first mating surface, the second member includes a second mating surface, the projection is defined in the first mating surface and the groove is defined in the second mating surface, and the first mating surface faces the

second mating surface and cooperates with the second mating surface to define the slot in the first state.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

In order that the disclosure may be well understood, there will now be described various forms thereof, given by way of example, reference being made to the accompanying drawings, in which:

FIG. 1 is a perspective view of a surgical instrument in accordance with the various teachings of the present disclosure.

FIG. 2 is an end view of a distal end of the surgical instrument in a first, collapsed state.

FIG. 3 is a side view of the distal end of the surgical instrument in the first, collapsed state.

FIG. 4 is a top view of the distal end of the surgical instrument in the first, collapsed state.

FIG. 5 is a perspective view of the distal end of the surgical instrument in a second, expanded state.

FIG. 6 is a side view of the distal end of the surgical instrument in the second, expanded state.

FIG. 7 is an end view of the distal end of the surgical instrument in the second, expanded state.

FIG. 8 is a cross-sectional view of the distal end of the surgical instrument in the second, expanded state taken along line 8-8 of FIG. 6.

FIG. 9 is a cross-sectional view of the surgical instrument, and a curved guide tube associated with the surgical instrument, which together form a kit, taken from the perspective of line 8-8 of FIG. 6.

FIGS. 10-14 schematically illustrate an exemplary method for soft tissue repair with the surgical instrument.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features. In addition, there is no intention to be bound by any expressed or implied theory presented in the preceding technical field, background, brief summary or the following detailed description. Further, the present disclosure may be practiced in conjunction with any method for soft tissue repair that would benefit from a surgical instrument that may be movable between states, and the use of the surgical instrument to perform a labral repair is merely one example according to the present disclosure. Further, it should be noted that many alternative or additional functional relationships or physical connections may be present in an example of the present

disclosure. In addition, while the figures shown herein depict an example with certain arrangements of elements, additional intervening elements, devices, features, or components may be present. It should also be understood that the drawings are merely illustrative and may not be drawn to scale.

As used herein, the term “axial” refers to a direction that is generally parallel to or coincident with an axis of rotation, axis of symmetry, or centerline of a component or components. For example, in a cylinder or disc with a centerline and generally circular ends or opposing faces, the “axial” direction may refer to the direction that generally extends in parallel to the centerline between the opposite ends or faces. In certain instances, the term “axial” may be utilized with respect to components that are not cylindrical (or otherwise radially symmetric). For example, the “axial” direction for a rectangular housing containing a rotating shaft may be viewed as a direction that is generally parallel to or coincident with the rotational axis of the shaft. Furthermore, the term “radially” as used herein may refer to a direction or a relationship of components with respect to a line extending outward from a shared centerline, axis, or similar reference, for example in a plane of a cylinder or disc that is perpendicular to the centerline or axis. In certain instances, components may be viewed as “radially” aligned even though one or both of the components may not be cylindrical (or otherwise radially symmetric). Furthermore, the terms “axial” and “radial” (and any derivatives) may encompass directional relationships that are other than precisely aligned with (e.g., oblique to) the true axial and radial dimensions, provided the relationship is predominantly in the respective nominal axial or radial direction. As used herein, the term “about”

denotes within 15% to account for manufacturing tolerances. In addition, the term “substantially” denotes within 15% to account for manufacturing tolerances.

As set forth in greater detail below, the present disclosure provides a surgical instrument, which can be used in an orthopedic surgical procedure involving the fixation of soft tissue to bone. The surgical instrument may generally include a distal end, which may be movable between a first, collapsed state and a second, expanded state (and positions in-between). In the first, collapsed state, the surgical instrument may define a substantially enclosed slot, which may retain at least a portion of a medical device, such as a flexible strand or suture. Generally, the surgical instrument may be configured to retain the medical device with at least about five pounds of force applied. By providing the surgical instrument movable between at least the first, collapsed state and the second, expanded state, the surgical instrument may be used within a surgical procedure without requiring a separate percutaneous insertion for the surgical instrument.

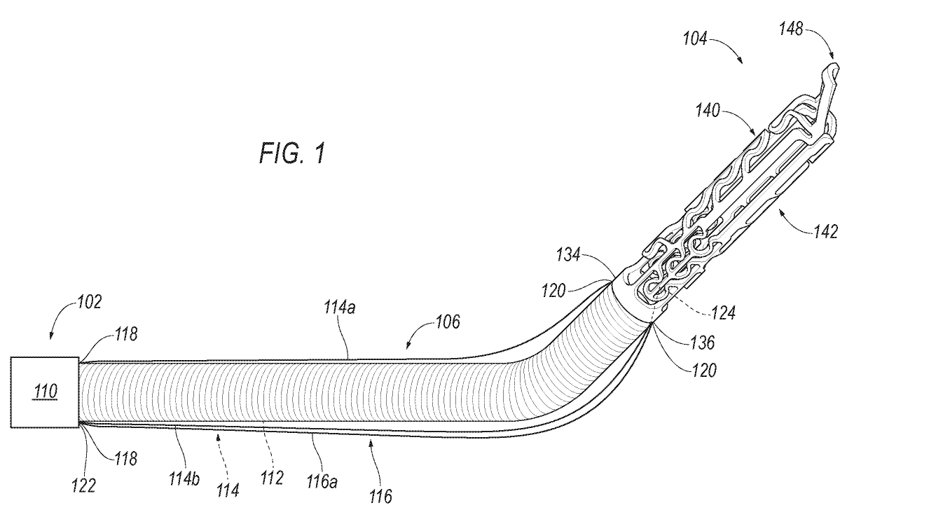

With reference to FIG. 1, a surgical instrument 100 is shown. In one example, the surgical instrument 100 may include a proximal end 102 opposite a distal end 104. A shaft 106 may extend from the proximal end 102 to the distal end 104. As will be described, the distal end 104 of the surgical instrument 100 may be movable between the first, collapsed state and the second, expanded state during a soft tissue repair surgical procedure, such as a labral repair, to assist in grasping and retaining a portion of a medical device, such as a flexible strand or suture, during the surgical procedure. Generally, by being movable between the first, collapsed state and the second, expanded state (and positions in-between), the surgical instrument 100 may enable a surgeon to perform the soft tissue repair without requiring an additional

percutaneous incision as the surgical instrument 100 may be guided through the anatomy with a smaller profile or diameter of the distal end 104, and moved into the second, expanded state with a larger profile or diameter of the distal end 104 once positioned at the predetermined location. It should be noted that while the surgical instrument 100 may be described herein as being using with a labral repair, the surgical instrument 100 may be used with any suitable surgical procedure, including, but not limited to Hill-Sach's repairs, transtendon repairs (for example, Partial Articular Surface Tendon Avulsion (PASTA) lesion repairs), fixation of soft tissue (ligament, tendon, graft, etc.) to bone, and for any soft tissue repairs including PASTA, rotator cuff, Achilles tendon, biceps and hip repairs, etc.

The proximal end 102 of the surgical instrument 100 may include a graspable portion 110. The graspable portion 110 enables the surgeon to control the surgical instrument 100 and manipulate the surgical instrument 100 when positioned at the predetermined location within the anatomy. The graspable portion 110 may also enable manipulation of the distal end 104 of the surgical instrument 100. The graspable portion 110 may have any desired shape to enable control and manipulation of the surgical instrument 100 and manipulation of the distal end 104 of the surgical instrument 100. The graspable portion 110 may be composed of a biocompatible material, for example, a biocompatible metal, metal alloy or polymer-based material. The graspable portion 110 may be manufactured through any suitable technique, including, but not limited to additive manufacturing. In one example, the proximal end 102 may be separately formed and coupled to the shaft 106, however, in other examples, the proximal end 102 may be integrally formed with the shaft 106.

In one example, the shaft 106 may be a flexible shaft, which interconnects the proximal end 102 to the distal end 104. The shaft 106 may be composed of a biocompatible material, for example, a biocompatible metal, metal alloy or polymer-based material. The shaft 106 may also be composed of a suitable shape-memory alloy, including, but not limited to nickel titanium or Nitinol. The shaft 106 may be manufactured through any suitable technique, including, but not limited to additive manufacturing, machining, wire cutting, extrusion, etc. In this example, the shaft 106 may be cylindrical and hollow to define a working channel or shaft lumen 112, but in other examples, the shaft 106 may have a different shape and may be solid. By providing the shaft 106 as a hollow shaft, additional surgical instruments, including, but not limited to, an imaging device, second surgical instrument, etc., may be inserted through the shaft lumen 112 to the predetermined location within the anatomy.

In one example, one or more steering wires 114 and/or one or more actuation wires 116 may extend along the shaft 106. Generally, each of the one or more steering wires 114 and one or more actuation wires 116 may extend from the proximal end 102 to or at the distal end 104 along an exterior surface of the shaft 106. In this example, the surgical instrument 100 may include two steering wires 114a, 114b. The steering wires 114a, 114b may be coupled to the proximal end 102, and may be arranged opposite each other along the exterior of the shaft 106 such that the steering wires 114a, 114b may be coupled on opposite sides of the distal end 104. Generally, each of the steering wires 114a, 114b may be manipulatable to pull and bend the shaft 106 in a predefined direction. Each of the steering wires 114a, 114b may include a proximal steering end 118 and a distal steering end 120. The proximal steering end 118 of each

of the steering wires 114a, 114b may be coupled to the graspable portion 110 to enable a surgeon to manipulate the respective steering wire 114a, 114b. For example, a manipulation of the proximal steering end 118 of the steering wire 114a may enable the shaft 106 to be pulled and bent in a first direction, which may be opposite a second direction that a manipulation of the proximal steering end 118 of the steering wire 114b may pull and bend the shaft 106. The distal steering end 120 of each of the steering wires 114a, 114b may be coupled to a respective portion of the distal end 104. In one example, a collar may be coupled to the exterior surface of the shaft 106, via laser welding, for example, and the distal steering end 120 of each of the steering wires 114a, 114b may be coupled to the collar. It should be noted that other techniques may be used to steer the surgical instrument 100, and the use of the steering wires 114a, 114b is merely an example. In certain instances, the exterior of the shaft 106, including the collar the steering wires 114a, 114b and the one or more actuation wires 116 may be coated or laminated.

In this example, the surgical instrument 100 may include one actuation wire 116a. In other examples, the surgical instrument 100 may include two actuation wires 116 or may not include any actuation wires 116. The actuation wire 116a may be coupled to the proximal end 102 and may be arranged to extend along the exterior of the shaft 106 to be coupled to the distal end 104. Generally, the actuation wire 116a may be manipulatable to move the distal end 104 between the first, collapsed state, the second, expanded state and positions between the first, collapsed state and the second, expanded state. The actuation wire 116a may include a proximal actuation end 122 and a distal actuation end 124. The proximal actuation end 122 of the actuation wire 116a

may be coupled to the graspable portion 110 to enable a surgeon to manipulate the actuation wire 116a. For example, a manipulation of the proximal actuation end 122 of the actuation wire 116a may enable the distal end 104 to move from at least the first, collapsed state to the second, expanded state and vice versa. The distal actuation end 124 of the actuation wire 116a may be coupled to a portion of the distal end 104. It should be noted that while the steering wires 114a, 114b and the actuation wire 116a are described herein as comprising wires, one or more of the steering wires 114a and the actuation wire 116a may be braided or coiled to provide additional structural support.

The distal end 104 may be coupled to the shaft 106. In one example, the distal end 104 may be discretely formed and coupled to the shaft 106, via adhesives, welding, etc., but in other examples, the distal end 104 may be integrally formed with the shaft 106. In this example, the distal end 104 may be composed of a biocompatible shape- memory, super-elastic or compliant metal, metal alloy or polymer-based material, including, but not limited to nickel titanium or Nitinol. The distal end 104 may be manufactured through any suitable technique, including, but not limited to additive manufacturing, casting, forging, extrusion, machining, etc. In one example, the distal end 104 may be composed of a nickel titanium or Nitinol tube, which may be extruded and machined via laser cutting, for example. In other examples, the distal end 104 may be composed of stainless steel, which may be extruded and machined via laser cutting.

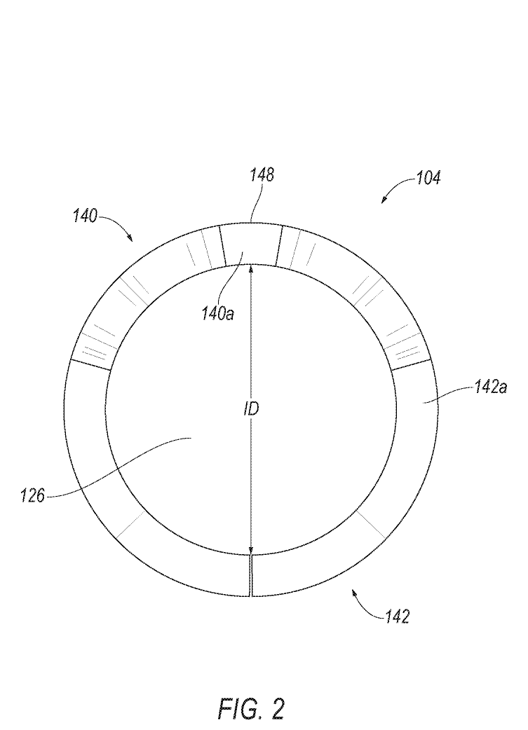

In one example, the distal end 104 of the surgical instrument 100 may include a first end 130 opposite a second end 132. In this example, with reference to FIG. 2, the first end 130 and the second end 132 may be open about the perimeter, and the distal end 104 may be defined as a hollow cylinder with a distal working channel

or distal lumen 126. The first end 130 may be coupled to the shaft 106. The first end 130 may also be coupled to the steering wires 114a, 114b. In one example, the steering wire 114a may be coupled to a first distal surface 134, and the steering wire 114b may be coupled to a second distal surface 136. The first distal surface 134 may be opposite the second distal surface 136. By coupling the steering wires 114a, 114b to opposite surfaces of the distal end 104, the surgical instrument 100 may be directed or steered in two different directions within the anatomy.

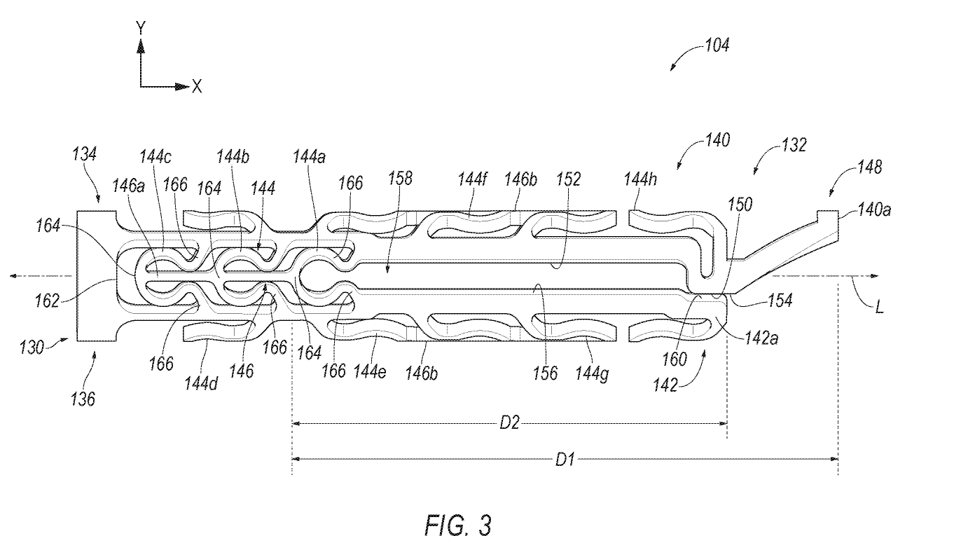

With reference to FIG. 3, a side view of the distal end 104 is shown. In FIG. 3, the distal end 104 is shown in the first, collapsed state. In this example, the distal end 104 may be defined to include a first portion or first member 140 and a second portion or second member 142, which extend along a longitudinal axis L of the distal end 104. The first member 140 and the second member 142 may cooperate to define a grasper. The first member 140 and the second member 142 may be defined at the second end 132 to extend toward the first end 130. The first member 140 may be opposite the second member 142. Generally, the first member 140 may be defined along the first distal surface 134, and the second member 142 may be defined along the second distal surface 136. As will be discussed, a plurality of struts 144 and a plurality of ribs 146 assist in moving the distal end 104 between the first, collapsed state and the second, expanded state.

In one example, the first member 140 may be defined to extend for a distance D1 along the longitudinal axis L from the second end 132 toward the first end 130, and the second member 142 may be defined to extend a second distance D2 along the longitudinal axis L from the second end 132 toward the first end 130. The second

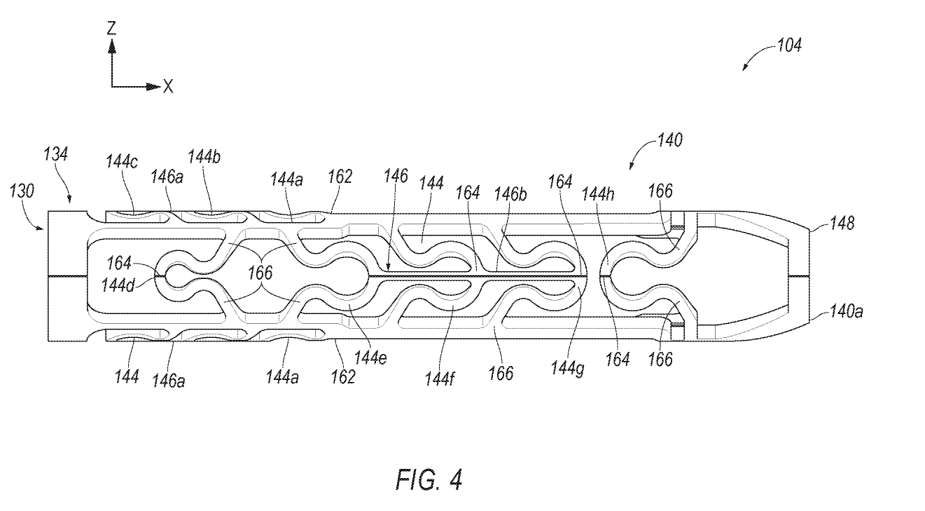

distance D2 may be different and less than the first distance D1. The first member 140 may include a flange or lip 148 at a first terminal end 140a. In this example, the lip 148 of the first member 140 may extend or project beyond a second terminal end 142a of the second member 142 to assist in guiding the distal end 104 within the anatomy. With reference to FIG. 4, the lip 148 may wrap around the first terminal end 140a of the first member 140 to provide structural support and to limit an expansion width of the distal end 104 during forming.

The first member 140 may also include a projection 150 and a recess 152. With reference back to FIG. 3, the projection 150 may be defined proximate the lip 148 so as to be positioned between the lip 148 and the recess 152. The projection 150 may extend outwardly from a first mating edge 154 and may contact a second mating edge 156 of the second member 142 when the first member 140 is in the first, collapsed state. The first mating edge 154 and the second mating edge 156 may be defined along the distal side surfaces 162 of the distal end 104, such that an interior or inner diameter of the distal end 104 may be open as shown in FIG. 2. Generally, with reference to FIG. 3, the first mating edge 154 may be a surface of the first member 140 that is defined to cooperate with the second mating edge 156 of the second member 142. The second mating edge 156 may be a surface of the second member 142 that faces the first member 140. In this example, the first mating edge 154 may face the second mating edge 156, and together, the first mating edge 154 and the second mating edge 156 may cooperate to enclose, capture or retain a portion of a medical device, such as a flexible strand or suture, in the first, collapsed state.

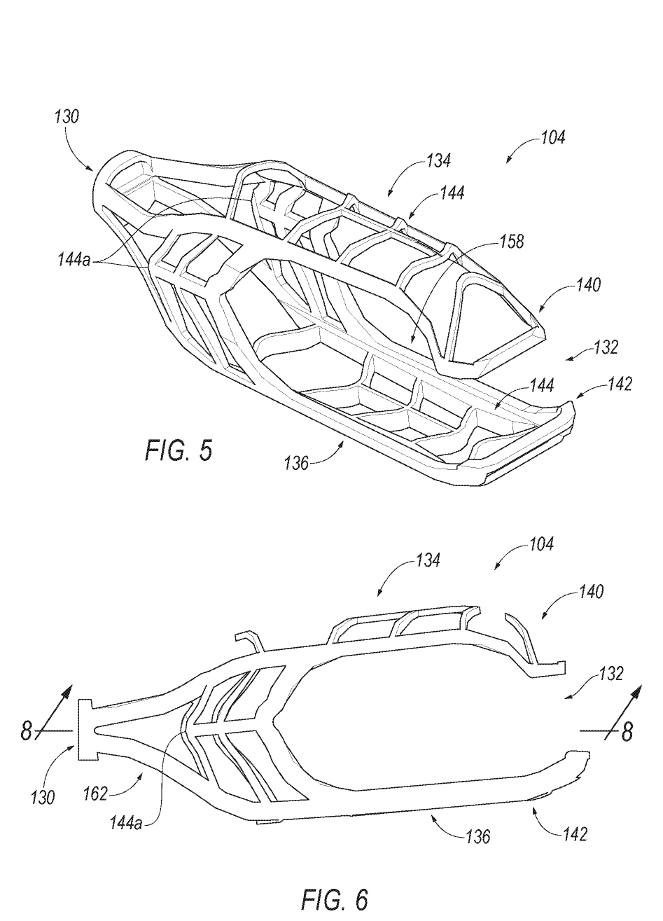

In this regard, the recess 152 may be defined along the first mating edge 154 such that the first mating edge 154 may extend axially inward from the projection 150. The recess 152 defined by the first mating edge 154 may cooperate with the second mating edge 156 to define a slot 158 in the first, collapsed state. The slot 158 may be enclosed proximate or near the first end 130 on distal side surfaces 162 of the distal end 104 by one of the struts 144 and may be enclosed proximate or at the second end 132 on the distal side surfaces 162 of the distal end 104 by contact between the projection 150 and the second mating edge 156. The distal side surfaces 162 are defined between the first distal surface 134 and the second distal surface 136. The contact between the projection 150 and the second mating edge 156 defines a terminal end of the slot 158. Generally, as will be described further herein, at least a portion of the first member 140 and the second member 142 may be in contact with each other or partially enclose the slot 158 in the first, collapsed state and may be spaced apart from each other in the second, expanded state (FIG. 5). Stated another way, in the first, collapsed state, the first member 140 and the second member 142 may extend substantially parallel to the longitudinal axis L, and in the second, expanded state, the first member 140 and the second member 142 may extend substantially oblique to the longitudinal axis L. It should be noted that while the first mating edge 154 and the second mating edge 156 surrounding the slot 158 are shown herein as being substantially smooth, in other examples, the first mating edge 154 and the second mating edge 156 along the slot 158 may include one or more teeth, ridges, projections, protrusions, etc. to assist in engaging and retaining the flexible strand and/or portions of the anatomy, if desired.

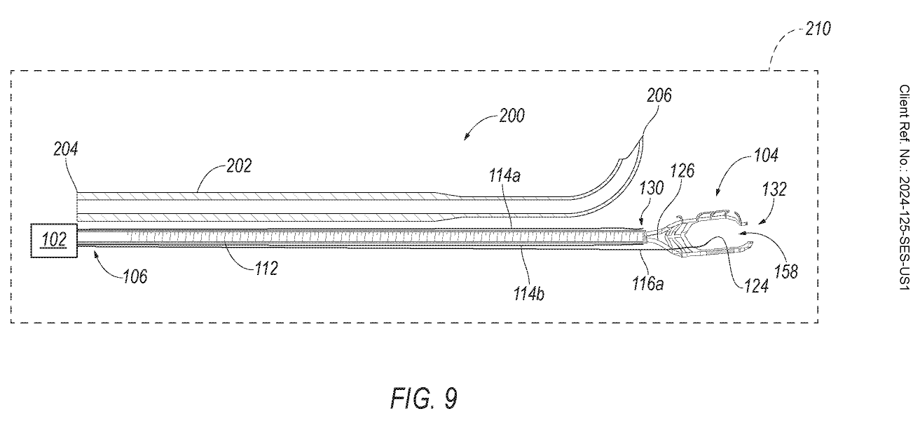

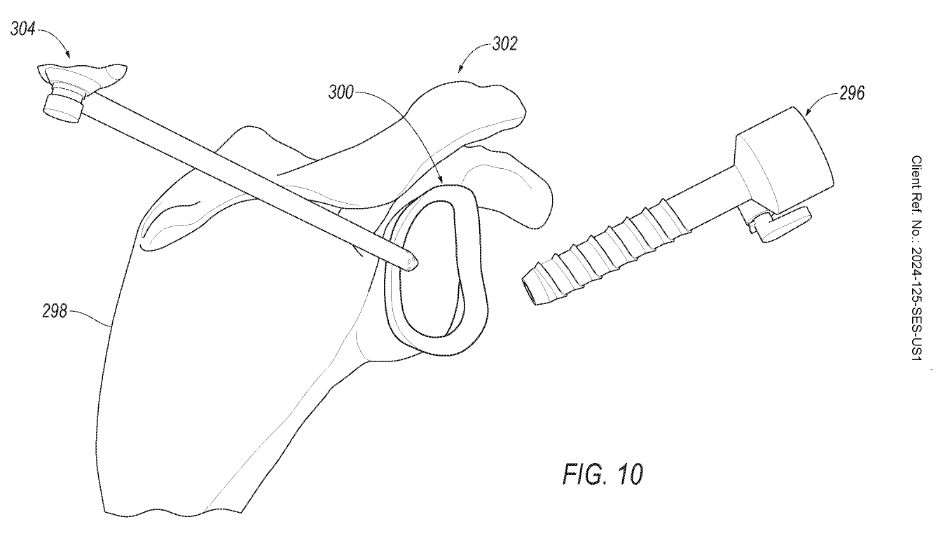

The second member 142 may include a groove 160 defined in the second mating edge 156 at the second terminal end 142a. The groove 160 may be sized and shaped to receive the projection 150 to enclose the slot 158 at or proximate the second end 132. The second member 142 may also be coupled to the actuation wire 116a. For example, the second member 142 may include a coupling post defined proximate the first end 130, which extends along an axis oblique to the longitudinal axis L. The actuation wire 116a may be coupled to the coupling post via crimping, tying, etc. By coupling the actuation wire 116a to the second member 142, a manipulation of the actuation wire 116a may cause the second member 142 to move relative to the first member 140 and transition the distal end 104 between the first, collapsed state and the second, expanded state. It should be noted that while the surgical instrument 100 may be described herein as including a single actuation wire 116a, in other examples, the surgical instrument 100 may include two actuation wires 116, with one actuation wire 116 coupled to each of the first member 140 and the second member 142. Further, while the actuation wire 116a may be described and illustrated herein as being coupled to the second member 142, in other examples, the actuation wire 116a may be coupled to the first member 140. In addition, the distal end 104 need not include the actuation wire 116a, and the distal end 104 may transition to from the first, collapsed state to the second, expanded state once advanced from a curved guide, such as curved guide 200 (FIG. 9), if desired. Moreover, the use of a coupling post or the like is merely an example, as any suitable technique may be used to couple the actuation wire 116a, if included, to the second member 142.

With reference back to FIG. 3, the struts 144 and the ribs 146 may be defined in the distal end 104 to support and enable the movement of the first member 140 and the second member 142 between the first, collapsed state and the second, expanded state. The struts 144 may be spaced apart along the distal end 104 and interconnected by respective ones of the ribs 146. In one example, each of the struts 144 may be substantially omega (Ω) in shape but may also be substantially C-shaped in the first, collapsed state. The struts 144 may generally include an apex 164, and a pair of legs 166 that define a base. By providing each of the struts 144 with a curvature, each of the struts 144 may elastically deform between the first, collapsed state and the second, expanded state. In the second, expanded state, the struts 144 may be substantially elongated and arcuate.

In this example, the distal side surfaces 162 of the distal end 104 may include about three of the struts 144a, 144b, 144c. The struts 144a, 144b, 144c may generally provide height expansion during a movement from the first, collapsed state to the second, expanded state or may enable expansion generally in the Y-direction. A pair of the struts 144a (FIG. 4) may define a hinge, and the pair of struts 144a may define an end of the slot 158. The first member 140 and the second member 142 may be hingedly coupled at the struts 144a and may be cantilevered from a remainder of the distal end 104 at the struts 144a to enable the movement of the second member 142 relative to the first member 140. In one example, the apex 164 of each of the struts 144a, 144b, 144c defined on the distal side surfaces 162 may be orientated in the same direction or may be orientated to face toward the first end 130. The legs 166 of the struts 144a, 144b, 144c may interconnect the first distal surface 134 and the second distal surface 136.

With reference to FIG. 4, the first distal surface 134 and the second distal surface 136 may include about five of the struts 144d, 144e, 144f, 144g, 144h. The struts 144d, 144e, 144f, 144g, 144h may generally provide width expansion during a movement from the first, collapsed state to the second, expanded state or may enable expansion generally in the Z-direction. In one example, the apex 164 of each of the struts 144d, 144h may be orientated in the same direction, and the apex of each of the struts 144e, 144f, 144g may be orientated in the same direction. The apexes 164 of the struts 144d, 144h may face toward the first end 130, and the apexes 164 of the struts 144e, 144f, 144g may face toward the second end 132. Thus, in this example, the apexes 164 of the struts 144d, 144e, 144f, 144g, 144h may not be orientated to face in the same direction but may be arranged to face in two or multiple directions to reinforce the first member 140 and the second member 142 during expansion. The legs 166 of the struts 144d, 144e, 144f, 144g, 144h interconnect the first mating edge 154 along the first distal surface 134 or the second mating edge 156 along the second distal surface 136.

With reference to FIGS. 3 and 4, the ribs 146 may reinforce the struts 144 and may provide structural stability to the struts 144. In one example, ribs 146a may interconnect and reinforce the struts 144a, 144b, 144c. Generally, the ribs 146a may be coupled to the struts 144a, 144b, 144c to extend through the apexes 164 of each of the struts 144a, 144b, 144c so as to be substantially parallel to the longitudinal axis L. The ribs 146b may interconnect and reinforce the struts 144d, 144e, 144f, 144g, 144h. Generally, the ribs 146b may be coupled to the struts 144d, 144e, 144f, 144g, 144h to extend through the apexes 164 of each of the struts 144d, 144e, 144f, 144g, 144h so as to be substantially parallel to the longitudinal axis L.

Generally, a remainder of the distal end 104 may be open about a perimeter of the distal end 104 such that one or more voids may be defined between adjacent ones of the struts 144, the ribs 146, the first mating edge 154 and the second mating edge 156. By providing the distal end 104 with voids, a weight of the distal end 104 may be reduced, and the voids may also assist in the movement of the distal end 104 between the first, collapsed state and the second, expanded state. In addition, it should be noted that the number of the struts 144 and the ribs 146 shown and described herein are merely exemplary, as the distal end 104 may include any number of the struts 144 and the ribs 146 as desired to transition the distal end 104 between the first, collapsed state and the second, expanded state.

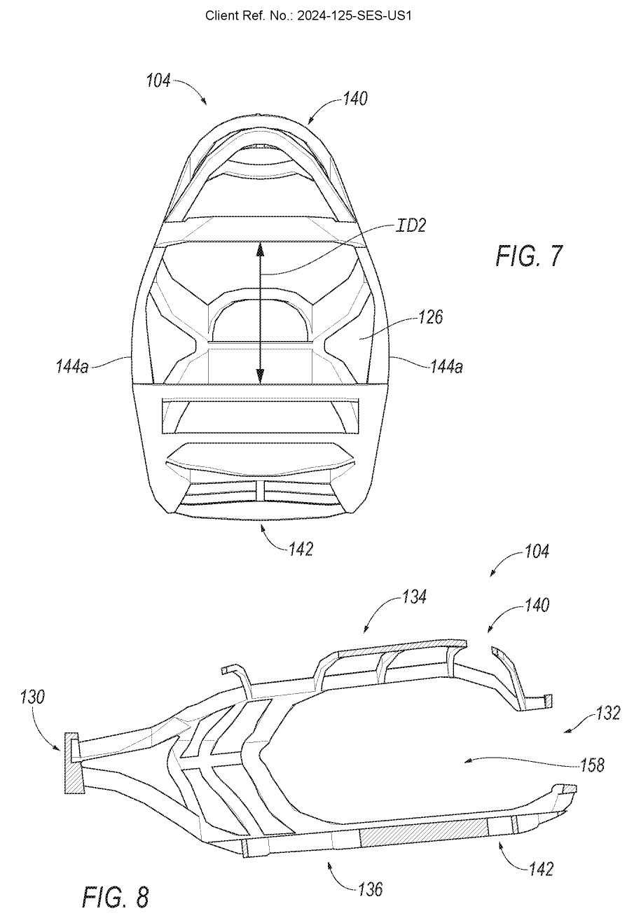

With reference to FIGS. 5 and 6, the distal end 104 is shown in the second, expanded state. In the second, expanded state, the first member 140 may be spaced apart from the second member 142. Generally, with reference to FIGS. 2 and 7, in the first, collapsed state, the second end 132 of the distal end 104 has a diameter ID, which may be different and less than a second diameter ID2 of the second end 132 in the second, expanded state. Stated another way, the distal lumen 126 may have the diameter ID at the second end 132 in the first, collapsed state, and the distal lumen 126 may have the second diameter ID2 in the second, expanded state. By providing the distal lumen 126 with the greater diameter ID2 in the second, expanded state, with reference to FIG. 8, the distal end 104 may be positioned about the flexible strand to ensure the flexible strand is surrounded by the distal end 104 as the distal end 104 moves from the second, expanded state to the first, collapsed state.

With reference to FIG. 9, the surgical instrument 100 may be inserted to the predetermined location within the anatomy with a curved guide 200. It should be noted that while the surgical instrument 100 is shown in the second, expanded state in FIG. 9, generally, the surgical instrument 100 may be guided through the curved guide 200 in the first, collapsed state. In one example, the curved guide 200 defines a guide lumen 202, which extends from a proximal guide end 204 to a distal guide end 206. A curvature or arc may be defined in the curved guide 200 proximate the distal guide end 206, which may assist in directing the surgical instrument 100 to the predetermined location within the anatomy. It should be noted that the curved guide 200 need not include the curvature or arc, if desired. Generally, the curved guide 200 may comprise any suitable curved guide 200 or guide for directing the surgical instrument 100 to the predetermined location within the anatomy. The surgical instrument 100 and the curved guide 200 may be packaged together in a kit 210, for example.

An example method using the surgical instrument 100 to fix soft tissue to bone to promote healing will be described below. It should be noted that the surgeon may or may not perform the method in the order shown and may perform the method partially or entirely. Moreover, the use of “surgeon” herein is meant to encompass other suitably trained staff that may assist with the method and use of the surgical instrument 100. The method described below may be performed during a surgical procedure.

Initially, the surgical instrument 100 may be assembled. With reference to FIG. 1, with the shaft 106 formed, the graspable portion 110 may be coupled to the shaft 106. The distal end 104 may be formed and heat-treated to define the first,

collapsed state and the second, expanded state, in the example of the distal end 104 being composed of a shape-memory metal alloy, such as nickel titanium or Nitinol. The steering wires 114a, 114b may be coupled to the first end 130 of the distal end 104, and the actuation wire 116a may be coupled to the second member 142. The distal end 104 may be coupled to the shaft 106, and the steering wires 114a, 114b and the actuation wire 116a may be positioned along the shaft 106. The steering wires 114a, 114b and the actuation wire 116a may be coupled to the graspable portion 110 to enable the surgeon to manipulate the surgical instrument 100 and the distal end 104.

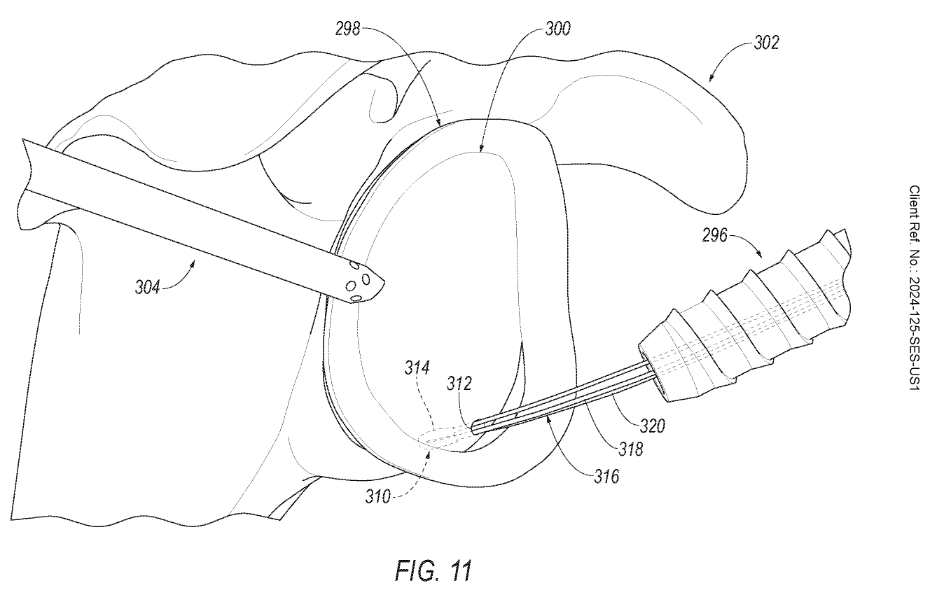

In the example of a labral repair, with reference to FIG. 10, a cannula 296 may be positioned at the predetermined location within the anatomy, which in this example, may be proximate the soft tissue or a labrum 298 associated with a glenoid 300 of a patient 302. In the example of FIG. 10, an imaging device 304, such as a camera, may be inserted percutaneously to the predetermined location within the anatomy to enable the surgeon to observe the labral repair. With reference to FIG. 11, in one example, the surgeon may employ an inserter to position a soft anchor 310, such as a FiberTak® RC Soft Anchor, commercially available from Arthrex, Inc. of Naples, Florida, USA through the cannula 296. The surgeon may form a bone hole 312 in the glenoid 300 with the inserter associated with the soft anchor 310 or may insert the curved guide 200 through the cannula 296 and use a drill inserted through the curved guide 200 to form the bone hole 312 to receive the soft anchor 310.

Generally, the soft anchor 310 may include a sheath 314 and at least one or the plurality of flexible strands 316. Generally, the sheath 314 may be a tubular sleeve made of a flexible material, such as a braided, woven, or knitted structure made

of biocompatible yarns, fibers, filaments, sutures or similar materials, or combinations of these materials. The sheath 314 may be free of barbs or protrusions and may be deformable to secure the soft anchor 310 to the anatomy. The sheath 314 may include a throughbore that receives the flexible strands 316, and includes at least one splice point or opening, which enables the flexible strands 316 to exit and re-enter into the throughbore.

The flexible strands 316 may be coupled to the sheath 314. In one example, the flexible strands 316 may be a suture, including, but not limited to FiberWire®, TigerWire®, or FiberChain® suture commercially available from Arthrex, Inc. of Naples, Florida, USA, although any type of suture may be utilized. The flexible strands 316 may also comprise suture tape, such as FiberTape® commercially available from Arthrex, Inc. of Naples, Florida, USA. Generally, the flexible strands 316 may comprise any soft, biocompatible flexible strands of material. In one example, the flexible strands 316 may include at least a repair suture 318 and a shuttle loop suture 320. The repair suture 318 may be the suture used to couple the soft tissue to the soft anchor 310, and the shuttle loop suture 320 may guide the repair suture 318 into engagement with the labrum 298.

With the soft anchor 310 deployed or set in the bone hole 312, the curved guide 200 may be inserted through the cannula 296, and the surgical instrument 100 may be inserted through the curved guide 200. The surgical instrument 100 may be moved from the first, collapsed state to the second, expanded state once positioned proximate the labrum 298. In the second, expanded state, the distal end 104 may be positioned about the repair suture 318, and moved from the second, expanded state to

the first, collapsed state to retain the repair suture 318 within the slot 158. With the repair suture 318 retained within the slot 158, the surgical instrument 100 may be retracted through the curved guide 200 to provide the surgeon with the repair suture 318.

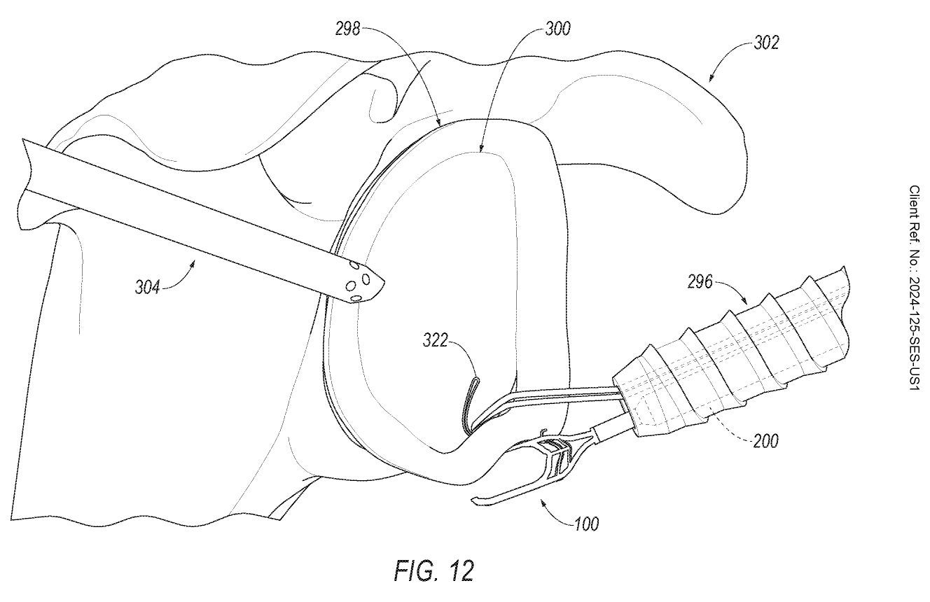

With reference to FIG. 12, the surgeon may insert a shape-memory metal or metal alloy loop, such as a nitinol loop 322, through the labrum 298. The surgical instrument 100 may be inserted through the curved guide 200 and moved from the first, collapsed state to the second, expanded state once positioned proximate the labrum 298. In the second, expanded state, the distal end 104 may be positioned about the nitinol loop 322, and moved from the second, expanded state to the first, collapsed state to retain the nitinol loop 322 within the slot 158. With the nitinol loop 322 retained within the slot 158, the surgical instrument 100 may be retracted through the curved guide 200 to provide the surgeon with the nitinol loop 322. The surgeon may pass the repair suture 318 through the nitinol loop 322. The nitinol loop 322 and the repair suture 318 may be coupled to the slot 158 of the surgical instrument 100 and the nitinol loop 322 and the repair suture 318 may be shuttled through the labrum 298.

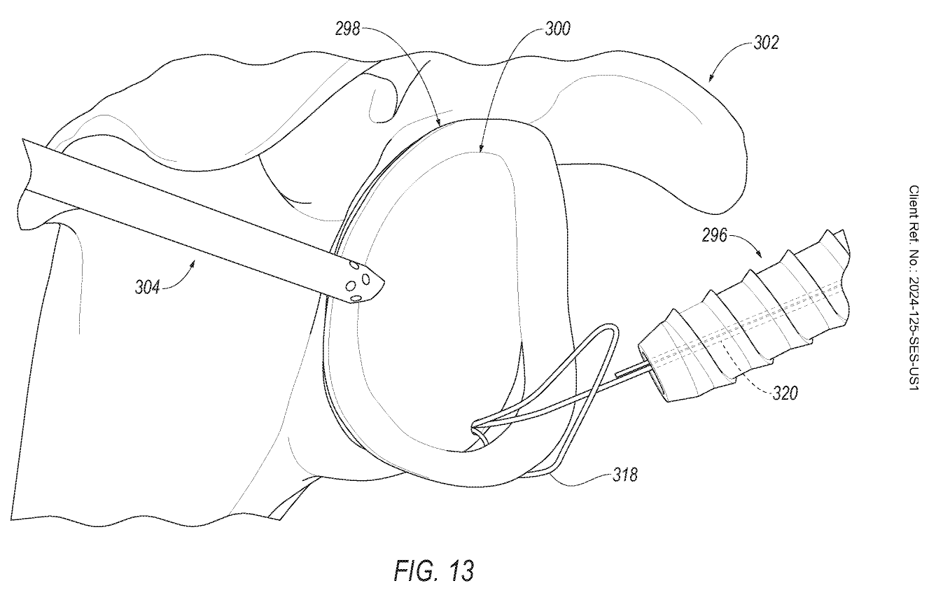

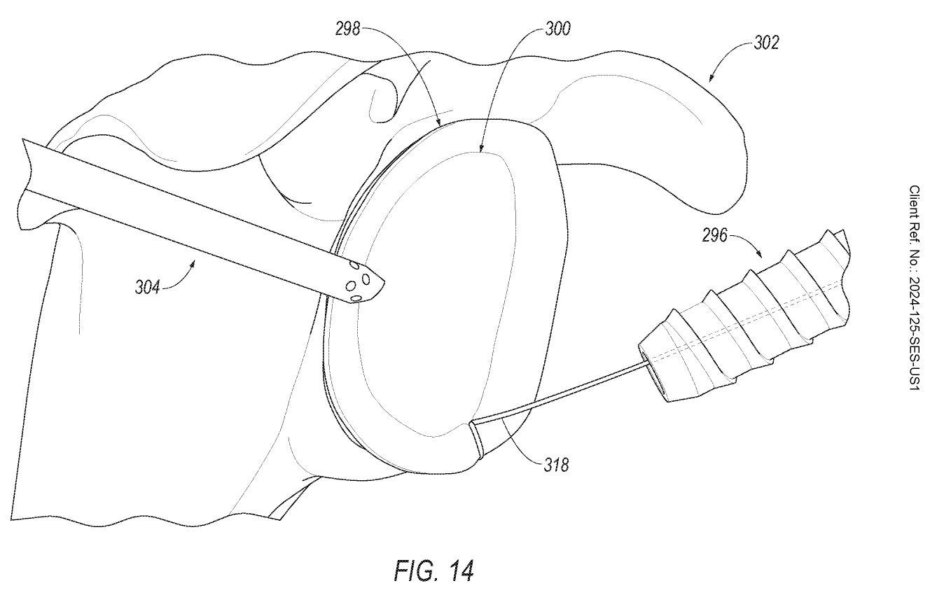

The surgical instrument 100 may be inserted through the curved guide 200 and moved from the first, collapsed state to the second, expanded state once positioned proximate the labrum 298 to retrieve the repair suture 318 and the shuttle loop suture 320. The surgical instrument 100 may be removed or retracted through the curved guide 200 while retaining the repair suture 318 and the shuttle loop suture 320. The repair suture 318 may be passed through the shuttle loop suture 320, and with reference to FIG. 13, the shuttle loop suture 320 may be pulled through the cannula 296 to pass the repair suture 318 through the sheath 314. With reference to FIG. 14, the repair suture 318 may

be tensioned to couple the labrum 298 to the bone hole 312 via the soft anchor 310 to repair the labrum. The repair suture 318 may be cut with a suture cutter. This process may be repeated to provide additional anchor points to secure the soft tissue to the bone, in this case, the labrum 298 to the glenoid 300.

Thus, by being movable between the first, collapsed state and the second, expanded state, surgical instrument 100 may be used in a surgical procedure to retrieve a medical device, such as the repair suture 318, the shuttle loop suture 320 and/or the nitinol loop 322, without requiring a separate percutaneous insertion. This may improve patient recovery time. Moreover, by being movable between the first, collapsed state and the second, expanded state (and positions in-between), the surgical instrument 100 may be positioned in the desired state to surround and grasp or retrieve the medical device. Further, by providing the surgical instrument 100 with the steering wires 114a, 114b, the surgical instrument 100 may be easily directed to the predetermined location and orientated to grasp or retrieve the medical device. In addition, the open shaft lumen 112 and distal lumen 126 may enable another medical instrument or device to be positioned within and inserted through the surgical instrument 100, such as an imaging device, a second surgical instrument or the like, which may further reduce a number of percutaneous incisions associated with the surgical procedure.

Unless otherwise expressly indicated herein, all numerical values indicating mechanical/thermal properties, compositional percentages, dimensions and/or tolerances, or other characteristics are to be understood as modified by the word “about” or "approximately" in describing the scope of the present disclosure. This modification is

desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A surgical instrument, comprising:
a proximal end; and
a distal end opposite the proximal end, the distal end including a tube that defines at least a first member and a second member, the first member and the second member movable between at least a first state and a second state, and in the second state the first member is spaced apart from the second member.

2. The surgical instrument of claim 1, wherein the first member cooperates with the second member to define a slot in the first state.

3. The surgical instrument of claim 2, wherein the first member has a lip at an end that extends beyond a second end of the second member.

4. The surgical instrument of claim 3, wherein the first member includes a projection proximate the lip, which is received within a groove defined in the second member in the first state to define a terminal end of the slot.

5. The surgical instrument of claim 4, wherein the first member includes a first mating surface, the second member includes a second mating surface, and the projection is defined in the first mating surface and the groove is defined in the second mating surface.

6. The surgical instrument of claim 5, wherein the first mating surface faces the second mating surface and cooperates with the second mating surface to define the slot in the first state.

7. The surgical instrument of claim 3, wherein the second end of the distal end is open about a perimeter of the distal end.

8. The surgical instrument of claim 2, wherein the distal end includes a plurality of struts, and at least a pair of the plurality of struts cooperate to enclose an end of the slot.

9. The surgical instrument of claim 8, wherein the distal end includes a plurality of ribs, with each rib associated with at least one strut of the plurality of struts.

10. The surgical instrument of claim 8, wherein each of the plurality of struts has an omega shape, with an apex and a pair of legs that define a base.

11. A surgical instrument, comprising:
a proximal end; and
a distal end opposite the proximal end, the distal end including a tube that defines at least a first member and a second member, the first member and the second member movable between at least a first state and a second state, the first member cooperates with the second member to define a slot in the first state and in the second state the first member is spaced apart from the second member.

12. The surgical instrument of claim 11, wherein the first member has a lip at an end that extends beyond a second end of the second member.

13. The surgical instrument of claim 12, wherein the first member includes a projection proximate the lip, which is received within a groove defined in the second member in the first state to define a terminal end of the slot.

14. The surgical instrument of claim 13, wherein the first member includes a first mating surface, the second member includes a second mating surface, and the projection is defined in the first mating surface and the groove is defined in the second mating surface.

15. The surgical instrument of claim 14, wherein the first mating surface faces the second mating surface and cooperates with the second mating surface to define the slot in the first state.

16. The surgical instrument of claim 12, wherein the second end of the distal end is open about a perimeter of the distal end.

17. The surgical instrument of claim 11, wherein the distal end includes a plurality of struts, and at least a pair of the plurality of struts cooperate to enclose an end of the slot.

18. The surgical instrument of claim 17, wherein the distal end includes a plurality of ribs, with each rib associated with at least one strut of the plurality of struts.

19. A surgical instrument, comprising:
a proximal end; and
a distal end opposite the proximal end, the distal end including a tube that defines at least a first member and a second member, the first member including a lip at an end that extends beyond a second end of the second member and a projection proximate the lip, the first member and the second member movable between at least a first state and a second state, the first member cooperates with the second member to define a slot in the first state with the projection received within a groove defined in the second member in the first state to define a terminal end of the slot and in the second state the first member is spaced apart from the second member.

20. The surgical instrument of claim 19, wherein the first member includes a first mating surface, the second member includes a second mating surface, the projection is defined in the first mating surface and the groove is defined in the second mating surface and the first mating surface faces the second mating surface and cooperates with the second mating surface to define the slot in the first state.
